(19) United States
(12) Patent Application Publication
(10) Pub. No.: US 2014/0066438 A1
(43) Pub. Date: Mar. 6, 2014

(54) DOPAMINE RECEPTOR D2 ANTAGONIST FOR PREVENTION AND TREATMENT OF FLAVIVIRUS INFECTION

(71) Applicant: Academia Sinica, Taipei City (TW)

(72) Inventor: YI-LING LIN, New Taipei City (TW)

(73) Assignee: Academia Sinica, Taipei City (TW)

(21) Appl. No.: 13/974,832

(22) Filed: Aug. 23, 2013

Related U.S. Application Data
(60) Provisional application No. 61/694,415, filed on Aug. 29, 2012.

(51) Int. Cl. A61K 31/5415; A61K 31/451

(52) U.S. Cl. CPC A61K 31/5415; A61K 31/451
USPC 514/225.5; 514/327

(57) ABSTRACT

Methods for preventing and/or treating flavivirus infection are disclosed. The method comprises administering to a subject in need thereof a composition comprising: a) a dopamine D2 receptor antagonist in an amount effective for preventing and/or treating flavivirus infection; and b) a pharmaceutically acceptable carrier. The dopamine D2 receptor antagonist may be selected from the group consisting of prochlorperazine or a salt thereof, and haloperidol.

### REFERENCES TO RELATED APPLICATION

The present application claims priority to U.S. Provisional Application Ser. No. 61/694,415, filed Aug. 29, 2012, which is herein incorporated by reference in its entirety.

### FIELD OF THE INVENTION

The present invention relates generally to antiviral compounds, and more specifically to dopamine D2 receptor antagonist as an antiviral agent against flavivirus infection.

### BACKGROUND OF THE INVENTION

Dengue virus (DENV) infection in humans causes a wide spectrum of illnesses ranging from mild dengue fever (DF) to severe dengue hemorrhagic fever (DHF) and life-threatening dengue shock syndrome (DSS). World Health Organization (WHO) estimates a prevalence of 50 to 100 million cases of DENV infection annually; however, a recent study suggested 390 million DENV infections per year.

DENV is enveloped and contains a positive-sense RNA genome encoding a polyprotein. By cellular and viral proteases, the polyprotein precursor is modified to yield mature structural capsid, membrane and envelope proteins as well as nonstructural NS1, NS2A, NS2B, NS3, NS4A, NS4B, and NS5 proteins. DENV infection initiates from binding to cell surface receptor(s) through the major structural envelope protein and enters via clathrin-mediated endocytosis. The internalized virions undergo acid-induced conformational changes and membrane fusion to release the viral genome. Translation of viral RNA produces proteins required for viral RNA replication through RNA-dependent RNA polymerization. The assembly of viral RNA and viral proteins generates the mature viral particle that is then released through the cellular secretary pathway.

Vaccines and antiviral drugs are two major means to control viral diseases, but DENV vaccines and anti-DENV drugs are not yet available.

### SUMMARY OF THE INVENTION

In one aspect, the invention relates to a method of preventing and/or treating flavivirus infection, comprising administering to a subject in need thereof a composition comprising: a) a dopamine receptor D2 antagonist in an amount effective for preventing and/or treating flavivirus infection; and b) a pharmaceutically acceptable carrier, wherein the dopamine receptor D2 antagonist is characterized as an antiviral agent having an activity in inhibiting flavivirus nonstructural protein 3 (NS3) production.

In another aspect, the invention relates to a method of preventing development of dengue hemorrhagic fever and/or dengue shock syndrome, comprising administering to a dengue fever patient a composition comprising: a) a therapeutically effective amount of dopamine receptor D2 antagonist; and b) a pharmaceutically acceptable carrier; wherein the dopamine receptor D2 antagonist is characterized as an antiviral agent having an activity in inhibiting dengue virus nonstructural protein 3 (NS3) production.

Further in another aspect, the invention relates to a method of preventing and/or treating dengue virus infection, comprising administering to a subject in need thereof a composition comprising: a) a dopamine receptor 2 antagonist in an amount effective for preventing and/or treating dengue virus infection; and b) a pharmaceutically acceptable carrier; wherein the dopamine receptor D2 antagonist is characterized as an antiviral agent having an activity in inhibiting dengue virus replication and/or inhibiting nonstructural protein 3 (NS3) production in a host cell.

Yet in another aspect, the invention relates to a method of inhibiting replication of flavivirus in a host, comprising administering to a subject in need thereof a dopamine receptor D2 antagonist in an amount effective for inhibiting replication of flavivirus, the dopamine receptor D2 antagonist being characterized as an antiviral agent having an activity in inhibiting flavivirus nonstructural protein 3 (NS3) production.

In one embodiment of the invention, the dopamine receptor D2 antagonist is at least one selected from the group consisting of prochlorperazine or a salt thereof, and haloperidol.

In another embodiment of the invention, the flavivirus is selected from the group consisting of a dengue virus, an encephalitis virus, and a West Nile virus.

In another embodiment of the invention, the encephalitis virus is a Japanese encephalitis virus.

In another embodiment of the invention, the subject is a high-risk human or a patient with fever before dengue diagnosis in epidemic areas during dengue outbreaks.

In another embodiment of the invention, the subject is a patient with dengue fever.

In another embodiment of the invention, the amount of prochlorperazine, or a salt thereof, is effective in inhibiting flavivirus binding to and/or entry into the cells of the subject.

In another embodiment of the invention, the amount of prochlorperazine, or a salt thereof, is effective in inhibiting flavivirus protein synthesis or flavivirus replication in the cells of the subject.

In another embodiment of the invention, the composition is a tablet, capsule or injection dosage form.

In another embodiment of the invention, the dengue virus is serotype 2 or serotype 1.

In another embodiment of the invention, the salt of prochlorperazine is selected from the group consisting of prochlorperazine maleate and prochlorperazine dimethanesulfonate.

Further in another embodiment of the invention, the administering step administers to a human prochlorperazine at least 0.4 mg/kg/day.

Yet in another embodiment of the invention, the administering step is performed by injection every 2 days for at least 10 days.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1 shows Prochlorperazine (PCZ) exhibits antiviral activity against DENV-2 infection. (A) PCZ cytotoxicity test. HEK293T cells were treated with solvent control or the indicated concentrations of PCZ for 24 h. AlamarBlue, XTT, and LDH assays were carried out to determine cell viability, cell proliferation, and cytotoxicity, respectively. Data are mean and standard deviation (SD) of 3 independent experiments. (B) HEK293T cells were infected with DENV-2 (multiplicity of infection [MOI]=0.1) in the absence (solvent) or presence of PCZ for 2 days. Representative immunofluorescence microscopy (400× magnification) of cells immunostained for DENV-2 NS3 (green) and DAPI for nuclei (blue). (C-D) HEK293T cells were infected with DENV-2 (MOI=1) in the absence (solvent) or presence of PCZ for 24 h. Western blot analysis of protein level of DENV-2 NS3 and α-tubulin for loading control; relative ratios of NS3 to α-tubulin are adjusted to solvent control (C). Plaque-forming assay of viral progeny production (plaque forming unit, PFU/ml) in culture supernatants of cells in panel C (D). Data are mean and SI) of 2 independent experiments. *P<0.05.

FIG. 2 shows PCZ inhibits DENV-2 entry into host cells. (A) HEK293T cells were adsorbed with lightning-Link Atto-488-labeled DENV-2 (green) (MOI=5) at 4° C. for 2 h. Solvent control or PCZ (15 and 20 μM) was added 15 min before the end of virus adsorption. After a washing with PBS, cells were then shifted to 37° C. for an additional 1 h incubation. Live-cell staining was performed before cell fixation to label the cell membrane with CellMask (red) and nuclei with Hoechst (blue). Representative confocal microscopy images are shown (630× magnification). (B) Plaque-forming assay of internalized DENV-2. BHK-21 cells were adsorbed with DENV-2 (MOI=5) at 4° C. for 2 h. After a washing with PBS, cells were shifted to 37° C. for an additional 1 h. Cells were physically destroyed to release the internalized DENV-2 and internalized virus was quantified by plaque-forming assays. Data are mean and SD of 2 independent experiments. *P<0.05 and **P<0.01.

FIG. 3 shows inhibition of DENV-2 entry by PCZ is associated with clathrin redistribution. HEK293 cells were adsorbed with Lightning-Link Atto-488-labeled DENV-2 (green) (MOI=10) at 4° C. for 2 h. Solvent control or PCZ (20 and 30 μM) was added 30 min before the end of virus adsorption. Cells were then shifted to 37° C. incubator for an additional 1 h. Cells were fixed, permeabilized and immunostained for clathrin heavy chain (red). Confocal microscopy of cytoplasmic distribution of clathrin and localization of labeled DENV-2 (630× magnification).

FIG. 4 shows PCZ interferes with DENV-2 binding onto the cells. (A) HEK293T cells pre-treated with the indicated doses of PCZ for 1 h were subsequently adsorbed with Lightning-Link Atto-488-labeled DENV-2 (green) (MOI=5) at 4° C. for 2 h in the absence (solvent) or presence of PCZ. Cells were then stained with CellMask for cell membrane (red) and Hoechst for nuclei (blue). Representative confocal microscopy images are shown (630× magnification). (B) Plaque-forming assay of cell-bound virus. A549 cells pretreated with PCZ for 1 h were adsorbed with DENV-2 (MOI=5) at 4° C. for 2 h and then physically destroyed to release the adsorbed DENV-2. The amount of cell-bound virus was determined by plaque-forming assays. Data are mean and SD of 2 independent experiments. *P<0.05 and **P<0.01.

FIG. 5 shows inhibition of DENV-2 binding by PCZ is associated with dopamine D2 receptor (D2R). (A) Antibody against D2R decreased DENV-2 binding onto cell surfaces. Lightning-Link Atto-488-labeled DENV-2 (green) (MOI=5) bound to N18 cells at 4° C. for 2 h with or without anti-D2R antibody (1:100 and 1:50 dilutions). Rabbit IgG antibody (1:50) was used as an antibody control. Confocal microscopy observation at 400× magnification. (B) The parental N18, D2R-knockdown (shD2R) and control knockdown (shLacZ) cells were adsorbed with Lightning-Link Atto-488-labeled DENV-2 (green) (MOI=10) at 4° C. for 2 h. Without permeabilization, cells were immunostained for D2R (red) and Hoechst for nuclei (blue). Representative confocal microscopy images are shown (630× magnification). (C) DENV-2 binding was reduced in D2R-deficient N18 cells. DENV-2 binding (MOI=5) was performed on the indicated cells at 4° C. for 2 h. Cells were then physically destroyed to release the adsorbed virus and cell-bound virus was quantified by plaque-forming assays. Data are mean and SD of two independent experiments. *P<0.05. (D) The inhibition of DENV-2 binding by PCZ depends on D2R. Cells were pre-treated with solvent of PCZ for 30 min, then adsorbed with DENV-2 (MOI=5) with or without PCZ at 4° C. for 2 h. After a washing with cold serum-free medium, cells were shifted to 37° C. for overnight incubation without PCZ. Western blot analysis of viral replication for DENV-2 NS3 and α-tubulin as a loading control.

FIG. 6 shows PCZ protects against DENV-2-induced lethality in an animal model. Stat1^{-/-} mice were challenged with 5×10⁴ PFU/mouse of DENV-2 NGC-N strain. The protective effect of PCZ dimaleate (A) and PCZ dimethanesulfonate (Novamin Injection) (B) were tested with immediate and 6-h delay treatment. (A) The mice received phosphate buffered saline (PBS; vehicle control, n=10), 5 mg (n=8) or 1 mg (n=8) PCZ-dimaleate/kg body weight/i.p. at the time of infection (immediate treatment), or 5 mg PCZ-dimaleate/kg body weight/i.p. at 6 h post-infection (6-h delay treatment, n=8). Mice received follow-up treatment with PBS (vehicle control) or half the dose of PCZ-dimaleate orally every 12 h until day-10 post infection. (B) Mice received PBS (vehicle control, n=5), 8 mg (n=4), 4 mg (n=5), or 2 mg (n=5) PCZ-Novamin/kg body weight/i.p. at the time of infection (immediate treatment); or 8 mg (n=5) or 4 mg (n=5) PCZ-Novamin/kg body weight/i.p. at 6 h post-infection (6-h delay treatment). Mice received follow-up treatment at 2-day intervals with the same dose of PCZ-Novamin i.p. until day 10 post-infection. The days with treatment are marked by arrows. Mice survival was checked daily and the data are presented as percentage of survival.

FIG. 7 shows PCZ-dimaleate exhibits antiviral activity against DENV-2 infection. (A) PCZ cytotoxicity test. HEK293T cells were treated with solvent control or the indicated concentrations of PCZ (5, 10, 15, 20 and 25 μM). AlamarBlue, XTT and LDH assays were carried out to determine cell viability, cell proliferation, and cytotoxicity, respectively. (B-D) HEK293T cells were infected with DENV-2 (MOI=0.1) in the absence (Solvent) or presence of PCZ (5, 10 and 15 μM) for 2 days. (B) Representative immunofluorescence microscopy imaging (400× magnification) of cells immunostained for DENV-2 NS3 (green) and DAPI for nuclei (blue), (C) Western blot analysis of protein levels of DENV-2 N53 and α-tubulin as a loading control; relative ratios of NS3 to α-tubulin adjusted to solvent control are shown. (D) Plaque-forming assay of viral progeny production (plaque forming unit, PFU/ml): Data are mean and SD of two independent experiments. **P<0.01. (E-F) PCZ-dimaleate shows antiviral effect against DENV-2 infection in baby hamster kidney BHK-21 and mouse neuroblastoma N18. BHK-21 cells (E) and N18 cells (F) infected with DENV-2 (MOI=0.1) were treated with or without PCZ for 2 days and 1 day, respectively. Western blot analysis of protein levels of DENV-2 NS3, actin or α-tubulin (loading control). Relative ratios of NS3 to actin or α-tubulin are adjusted to solvent control.

FIG. 8 shows endogenous D2R expression in cell lines with or without DENV-2 infection. (A) Western blot analysis of protein levels of D2R and actin (loading control) in HEK293T, A549, hepatocellular carcinoma HepG2, and neuroblastoma SH-SY5Y cells. (B) Western blot analysis of protein levels of D2R and actin (loading control) in A549 cells infected with DEN-V-2 (MOI=5).

FIG. 9 shows PCZ exhibits antiviral effects against DENV-1 and JEV infection. (A) PCZ dimethanesulfonate (Novamin injection) reduced DENV-1 replication. HEK293T cells infected with DENV-1 (MOI=0.1) were treated with or without the indicated doses of PCZ for 2 days. Western blot analysis of protein levels of DENV-1 NS3 and α-tubulin (loading control). (B) PCZ-dimaleate inhibits Japanese encephalitis virus (JEV) infection. BHK-21 cells were infected with JEV (MOI=0.1) in the absence or presence of PCZ for 2 days. Western blot analysis of protein levels of JEV NS3 and actin (loading control). Relative ratios of NS3 to actin or α-tubulin adjusted to solvent control are shown.

FIG. 10 shows PCZ does not likely affect DENV-2 translation and RNA replication. DENV-2 replicon was constructed to contain the SP6 promoter upstream of a Renilla luciferase gene flanked by the DENV 5′ UTR plus a portion of the core gene in the 5′ end; portions of the envelope gene, full length of non-structural genes and 3′ UTR in the 3′ end. In vitro transcription was performed to generate the replicon RNA. HEK293T cells were co-transfected with replicon RNA and control firefly luciferase RNA. PCZ (10 μM) was applied to the transfected cells at 5 h post-transfection. Cells were then harvested at the indicated times for dual luciferase assays. Data are mean and SD of relative renilla luciferase to firefly luciferase from two independent experiments. No significant difference between solvent and PCZ-treated cells at each time point.

FIG. 11 shows haloperidol, another D2R antagonist, exhibits antiviral effects against DENV-2 infection. HEK293T cells were infected with DENV-2 (MOI=0.1) with or without haloperidol for 1 day. Western blot analysis of protein levels of DENV-2 NS3 and α-tubulin (loading control). Relative ratios of NS3 to α-tubulin adjusted to solvent control are shown.

### DETAILED DESCRIPTION OF THE INVENTION

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The term “treating” or “treatment” refers to administration of an effective amount of the compound to a subject in need thereof, who has flavivirus infection, or a symptom or predisposition toward such a disease, with the purpose of cure, alleviate, relieve, remedy, ameliorate, or prevent the disease, the symptoms of it, or the predisposition towards it. Such a subject can be identified by a health care professional based on results from any suitable diagnostic method.

“An effective amount” refers to the amount of an active compound that is required to confer a therapeutic effect on the treated subject. Effective doses will vary, as recognized by those skilled in the art, depending on rout of administration, excipient usage, and the possibility of co-usage with other therapeutic treatment.

Flavivirus is a genus of the family Flaviviridae. This genus includes the West Nile virus, dengue virus, tick-borne encephalitis virus, yellow fever virus, and several other viruses which may cause encephalitis. The flavivirus does not include hepatitis C virus.

The terms “dopamine receptor D2”, also known as D2R, and “dopamine D2 receptor” are interchangeable.

The invention relates to the discovery that prochlorperazine (PCZ), a dopamine D2 receptor (D2R) antagonist approved to treat nausea and vomiting in humans, has potent in vitro and in vivo antiviral activity against DENV infection. Measurement of viral protein expression and viral progeny production revealed greatly reduced infection with DENV serotype 2 (DENV-2) in cells treated with noncytotoxic doses of PCZ, with a half-maximal effective concentration of 0.0884 μM. PCZ appears to block DENV-2 infection by targeting viral binding and viral entry via D2R- and clathrin-dependent mechanisms, respectively. In an animal model, PCZ treatments, given immediately or 6 h after DENV-2 infection of Stat1 deficiency mice, completely protected against or delayed mortality. Overall, PCZ showed a previously unknown antiviral effect against DENY infection. Prophylactic and/or therapeutic treatment with PCZ might reduce viral replication and relieve clinical symptoms of DENV infection in humans.

PCZ may block DENV-2 infection by antagonizing DENV-2 binding to D2R, implying that D2R might serve as a receptor for DENV-2 infection. Three lines of evidences support this notion: 1) Fluorescently labeled DENV-2 co-localized with D2R on cell surface 2) PCZ reduced the levels of DENV-2 bound on cell surface; and 3) Lower DENV-2 binding was noted on D2R knockdown cells. PCZ has also been found to block clathrin-dependent endocytosis that is known to involve in DENY entry. We tested whether viral entry, besides viral binding, is also affected by PCZ. It was found that clathrin distribution was relocated in cells treated with PCZ and the levels of DENV entry were reduced by PCZ treatment. Thus, PCZ could block DENV infection by interfering with DENY binding to D2R and also DENV entry through clathrin-dependent endocytosis. Furthermore, we used a STAT1−/− mice challenge model to assess whether PCZ is able to alleviate the outcome of DENV infection in vivo.

Oral administration of PCZ at 5 μg/gram body weight daily completely protected the DENV-2 challenge mice, whereas more than 70% of the mice given solvent control succumbed to DENV-2 infection. Overall, the FDA-approved drug PCZ has a previously unknown antiviral function and could be further evaluated as a potential therapeutic strategy against DENV infection. In addition, this study also reveals dopamine receptor as a potential DENV receptor which, in turn, could provide a rationale to design small molecules to block DENY infection.

### EXAMPLES

### Methods

Viruses, Cell Lines, and Drugs.

DENV-2 PL046 strain (Genbank accession: AJ968413.1) isolated from a Taiwan dengue fever patient was used for in vitro study. DENV-2 New Guinea C-N (NGC-N) strain, kindly provided by Dr. Ching-Juh Lai (US National Institutes of Health), was used for the animal study. Viruses were propagated in C6/36 mosquito cells and titrated by plaque-forming assay in BHK-21 cells as described. The human embryonic kidney (HEK) 293T/17 cell line (ATCC, CRL-11268) was grown in DMEM containing 10% fetal bovine serum (FBS). The human lung epithelial carcinoma A549 cell line (ATCC, CCL-185) was cultured in F-12 medium (INVITROGEN™) supplemented with 10% FBS. Baby hamster kidney BHK-21 cells (ATCC: CCL-10) and mouse neuroblastoma N18 cells were cultured in RPMI 1640 medium (GIBCO®) supplemented with 5% FBS. Prochlorperazine dimaleate (P9178) and haloperidol (H1512) were from Sigma-Aldrich. Prochlorperazine dimethanesulfonate (Novamnin Injection) was from Taiwan Shionogi Co.

Drug Cytotoxicity Assays.

Cells treated with the indicated doses of PCZ were analyzed by use of the Cytotoxicity Detection Kit (LDH) (Roche), Cell Proliferation Kit (XTT) (Roche) and ALAMARBLUE® Cell Viability Assay (Life Technologies). Briefly, cells treated with PCZ at the indicated doses for overnight underwent XTT, LDH and ALAMARBLUE® assay. Sample absorbance from XTT and LDH assays was determined by use of an ELISA reader (Molecular Devices) at 450 and 490 nm, respectively. For AlamarBlue viability assay, fluorescence intensity was monitored at 570 nm excitation and 585 nm emission by use of a microplate reader (Molecular Devices).

Cell-Based Antiviral Assay.

Monolayers of cells (HEK293T, BHK-21 and N18) in 6- or 12-well plates were infected with DENV-2 at the indicated multiplicity of infection (MOI). Virus was adsorbed for 2 h with the indicated concentrations of PCZ, then cells were washed thoroughly to remove unbound viruses and incubated for an additional 24 to 48 h with or without PCZ. The antiviral effect of PCZ was evaluated by immunofluorescence assay (IFA), immunoblotting and plaque-forming assay.

Preparation of Fluorescent-Labeled DENV-2.

DENV-2 was purified and labeled with Lightning-Link Atto-488 (Innova Biosciences) as previously described. Briefly, virus was ultracentrifugated through a 35% sucrose cushion at 35,000 rpm in a Beckman SW41 rotor for 3.5 h at 4° C. The viral pellets were resuspended in PBS and labeled with Lightning-Link Atto-488 according to the manufacturer's guidelines (Innova Biosciences). Level of virus titer was determined by plaque-forming assay.

Virus Binding Assay.

Monolayers of HEK293T and A549 cells were pretreated or not with PCZ for 1 h, then were adsorbed with DENV-2 (MOI=5) for 2 h at 4° C. with rocking on a linear shaker with or without PCZ. For confocal microscopy, fluorescent-labeled viruses were used and the cells were stained with 5 μg/ml of Cell Mask Deep Red Plasma Membrane Stain (Life Technologies) and Hoechst (1:2000) for 30 min at 4° C. before the end of virus adsorption. Cells were washed with cold phosphate buffered saline (PBS), fixed with cold 4% paraformaldehyde (PFA) for 10 min on ice, washed twice with cold 0.1 μM glycine, then extensively washed with PBS. For quantification by plaque-forming assay, cells were adsorbed with unlabeled DENV-2 (MOI=5) for 2 h at 4° C. After extensive washing with cold washing buffer (0.2% bovine serum albumin in PBS, pH 6.8) and PBS, cells were harvested with use of cell scrapers, and the adsorbed viruses were released by passing the cells through a 27-G needle 3 times. Cell lysates were centrifuged at 12,000 rpm for 1 min, and the supernatant was used to determine the amount of cell-bound virus by plaque-forming assay.

Virus Entry Assay.

Monolayers of HEK293T and BHK-21 cells were adsorbed with DENV-2 (MOI=5) for 2 h at 4° C. on a linear shaker in the absence of PCZ. At 15 min before the end of virus adsorption, PCZ was added to the cell cultures. Cells were gently washed once with cold PBS, then were shifted to a 37° C. CO₂ incubator for an additional 1 h to allow virus entry with or without PCZ. For confocal microscopy, fluorescent-labeled viruses were used and cells were stained with CellMask (5 μg/mL) and Hoechst (1:2000) for 30 min at 37° C. Cells were washed 3 times with cold washing buffer and once with cold PBS, then were fixed with 4% PFA for 10 min on ice. Cells were washed twice with cold 0.1 μM glycine, then PBS. For quantification of internalized viruses, after extensive washing with cold PBS, cells were harvested with use of cell scrapers and passed through a 27-G needle 3 times. Level of virus titers was determined by plaque-forming assay.

Antibody Competition Assay.

N18 cells were adsorbed with fluorescent-labeled DENV-2 (MOI=5) for 2 h at 4° C. with or without rabbit anti-D2R antibody (1:50 and 1:100; Santa Cruz Biotechnology, sc-9113). Rabbit-IgG antibody (1:50) was used as a non-specific IgG control. For confocal microscopy, cells were incubated with Hoechst (1:2000) for 15 min at 4° C. before the end of virus adsorption. Cells were then washed extensively with washing buffer and fixed with 4% PFA.

Lentivirus Preparation and D2R-Knockdown.

The lentivirus vector pLKO.1 carrying a short hairpin RNA (shRNA) targeting the mouse dopamine D2 receptor (5′-CCACTACAACTACTATGCCAT-3′, SEQ ID NO: 1) or LacZ (5′-TGTTCGCATTATCCGAACCAT-3′, SEQ ID NO: 2) from the Taiwan National RNAi Core Facility was cotransfected with pMD.G and pCMVΔR8.91 into HEK293T cells by use of Lipofectamine 2000 (INVITROGEN™). The lentiviruses were harvested from culture supernatants and concentrated by ultracentrifugation at 35,000 rpm in a Beckman SW41 rotor for 3.5 h at 4° C. The viral pellets were resuspended and used to transduce N18 cells. The D2R-deficient N18 cells (shD2R-N18) and LacZ-control cells (shLacZ-N18) were selected with puromycin (5 μg/ml).

Western Blot Analysis.

Cells were lysed with RIPA buffer (10 mM Tris, pH 7.5, 5 mM EDTA, 150 mM NaCl, 0.1% SDS, 1% TritonX-100, and 1% sodium deoxycholate) containing a cocktail of protease and phosphatase inhibitors (Roche). The protein concentration was measured by the Bio-Rad Dc protein assay (Bio-Rad). Equal amounts of proteins were separated by SDS-PAGE and transferred to a nitrocellulose membrane (Hybond-C Super; Amersham/GE Healthcare), which was blocked with 5% skim milk in PBS with 0.1% TWEEN® 20 and incubated with the primary antibodies anti-DENV NS3 (49), anti-JEV NS3, anti-D2R (Santa Cruz Biotechnology, sc-9113), anti-β-actin (CHEMICON®), or anti-α-tubulin (Sigma-Aldrich), then horseradish peroxidase-conjugated secondary antibody (Jackson ImmunoResearch); signals were detected by enhanced chemiluminescence (ECL, Pierce).

Immunoflourescence Assay (IFA) and Confocal Imaging.

Cells were fixed with 4% PFA and permeabilized with 0.5% Triton X-100 in PBS. After a blocking with 5% skim milk in PBS, DENV protein expression was detected in cells incubated with mouse anti-DENV-2 NS3 antibody (49), then Alexa Fluor-488-conjugated anti-mouse secondary antibody (Molecular Probes) for 1 h at room temperature. The nuclei were stained with DAPI or Hoechst (Molecular Probes). Cells were examined under an inverted fluorescence microscope (Olympus 1X71). For confocal microcopy, cells were seeded on coverslips in 24-well culture plates. After virus absorption and treatment, cells were extensively washed with cold washing buffer and PBS, then fixed with cold 4% PFA. Cells were washed twice with cold 10 mM glycine solution, then PBS. For clathrin staining, cells were permeabilized with 0.5% Triton X-100 in PBS and blocked with 5% BSA in PBS. Cells were stained with mouse anti-clathrin heavy chain (X22) (Pierce, MA1-065), then anti-mouse Alexa Fluor-568-conjugated secondary antibody (Molecular Probes). The coverslip was mounted onto a slide and examined under confocal laser scanning microscope (ZEISS LSM 700).

Animal Study.

The mouse experiments were approved and performed in accordance with the guidelines of the Academia Sinica Institutional Animal Care and Utilization Committee. Groups of 5-week-old Stat1-deficient (SaT1^{-/-}) mice were challenged intraperitoneally (i.p.) with 5×10⁴ plaque-forming units (PFU)/mouse of DENV-2 NGC-N strain and simultaneously injected with 30 μl PBS intracranially (i.c.) into the right hemisphere of the brain. To study the efficacy of PCZ-dimaleate administered mainly by an oral route, mice were divided into 4 groups for treatment: PBS (vehicle control); 5 or 1 mg of PCZ/kg body weight/i.p. at the time of infection (immediate treatment); or 5 mg of PCZ/kg body weight/i.p. at 6 h post-infection (6-h delay treatment). Thereafter, twice a day, mice received a half dose of the first treatment orally with PBS (vehicle control) or 2.5 or 0.5 mg of PCZ/kg body weight/oral/12 h up to day 10 post-infection. To study the effect of PCZ-Novamin, mice were divided into 6 groups for treatment: PBS (vehicle control); 8, 4 or 2 mg of PCZ/kg body weight/i.p. at the time of infection (immediate treatment); or 8 or 4 mg of PCZ/kg body weight/i.p. at 6 h post-infection (6-h delay treatment). Thereafter, every 2 days, mice received PBS or the same dose of PCZ up to day 10 post-infection. The mice were checked daily for mortality.

Statistical Analysis.

Data are presented as mean±standard deviation (SD) and were compared by two-tailed Student's t-test. Statistical significance was set at p<0.05 and p<0.01. Survival was descriptively analyzed by use of SigmaPlot 10.0 (Systat Software Inc.). For immunoblotting, the band density was quantified by using ImageJ software (NIH).

### Results

PCZ Exhibits Antiviral Effects Against DENV-2 Infection.

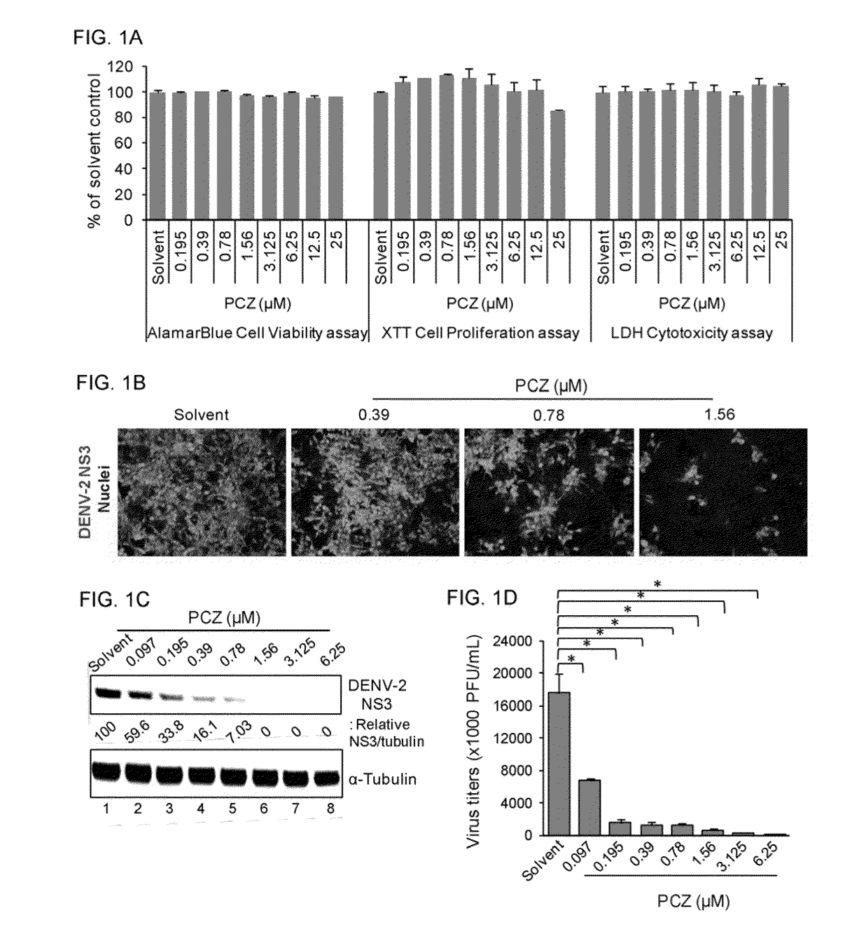

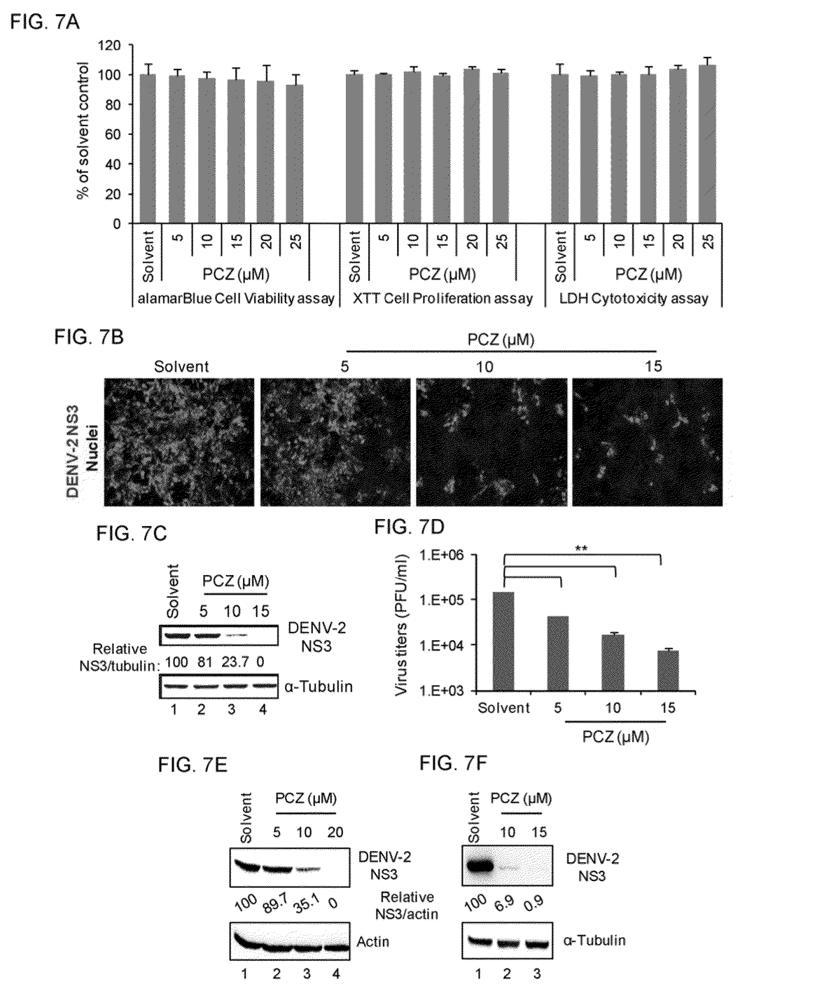

To evaluate the antiviral potential of PCZ against DENV infection, we first determined the noncytotoxic doses of a clinically used PCZ, prochlorperazine dimethanesulfonate (Novamin Injection) (PCZ-Novamin). Treating HEK293T cells with PCZ up to 25 μM did not significantly affect cell viability, cell proliferation, or cytotoxicity (FIG. 1A). We then tested whether treatment with noncytotoxic PCZ inhibited DENV-2 by measuring viral protein expression and viral progeny production. The protein expression of viral NS3 was dose-dependently reduced with PCZ treatment in DENV-2-infected HEK293T cells (FIGS. 1B and 1C). PCZ treatment also significantly reduced viral progeny production as measured by plaque-forming assay (FIG. 1D). The half-maximal effective concentration (EC₅₀) of PCZ against DENV-2 infection was calculated as 0.0884 μM based on a published method. This concentration is far below the cytotoxic dose of PCZ, because cells treated with 25 μM of PCZ showed no cytotoxic effects (FIG. 1A). The anti-DENV-2 effect of PCZ was also confirmed by treating HEK293T as well as baby hamster kidney BHK-21 and mouse neuroblastoma N18 cells with a PCZ solution prepared from prochlorperazine dimaleate salt (PCZ-dimaleate) (FIG. 7). Thus, at noncytotoxic concentrations, PCZ shows antiviral effects against DENV-2 infection.

PCZ Inhibits DENV-2 Entry.

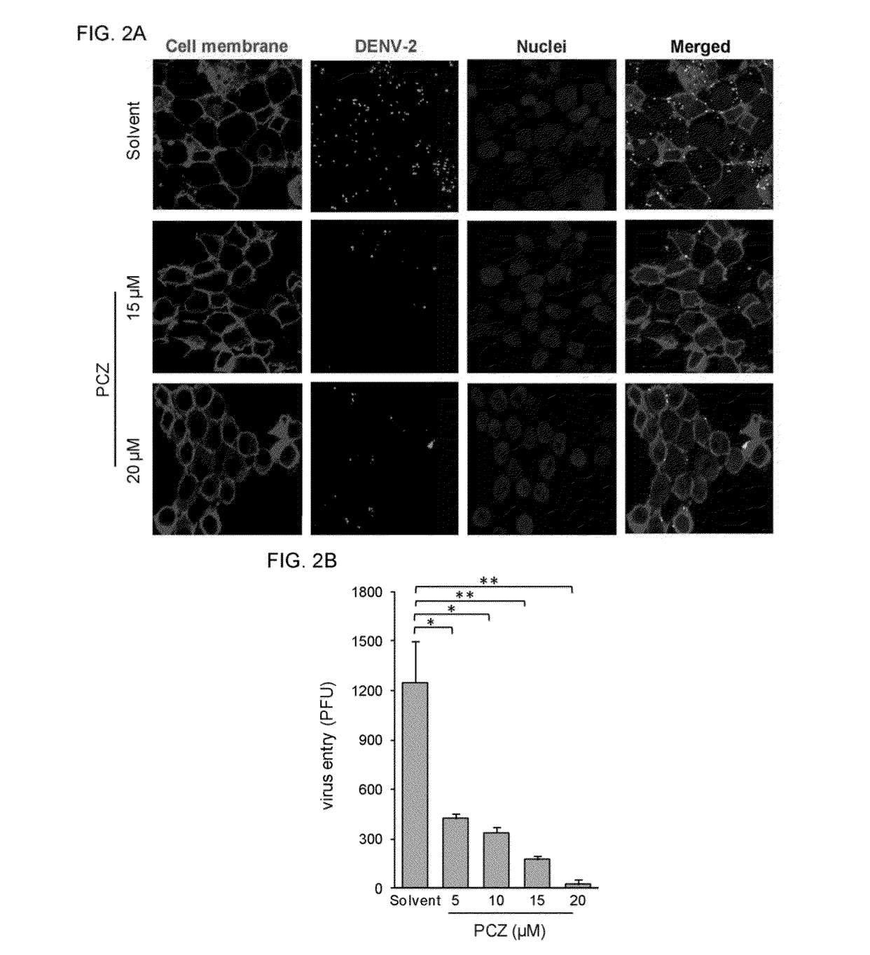

To determine the antiviral mechanism of PCZ, we investigated whether PCZ could block DENV entry. We performed a viral entry assay by allowing fluorescent-labeled DENV-2 to bind to cell surface at 4° C. for 2 h, then shifting the cells to 37° C. for viral internalization in the presence or absence of PCZ. Without PCZ treatment, after 1 h of incubation at 37° C., most of the fluorescent-labeled DENV-2 entered cells and were seen in cytosol on confocal microscopy (FIG. 2A). However, with PCZ treatment, the amounts of DENV-2 were greatly reduced in the intracellular compartment, so PCZ may inhibit DENV entry. Consistent with the confocal microscopy result, PCZ significantly reduced the levels of DENV-2 internalization quantified by plaque forming assay (FIG. 2B).

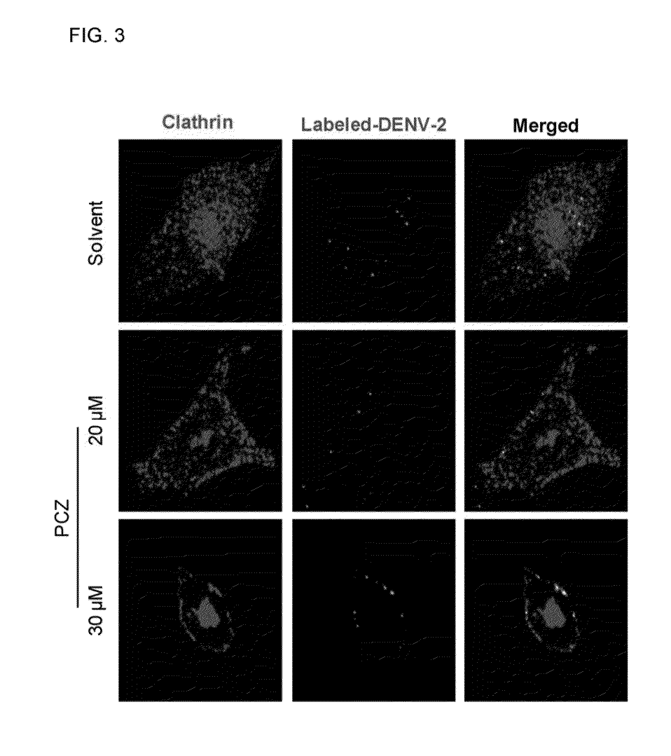

Clathrin is responsible for receptor internalization, regulation of signal transduction and synaptic vesicle recycling by its forming a lattice-like coat vesicle. Chlorpromazine can affect clathrin distribution and relocation, so we tested whether the inhibitory effect of PCZ on DENY entry was related to clathrin distribution. We performed viral entry assay with fluorescent-labeled DENV-2 and examined the cellular distribution of clathrin upon PCZ treatment. As compared to the solvent-treated cell, cells treated by PCZ showed a distinct clathrin distribution (FIG. 3), indicating that PCZ could cause cellular clathrin relocation. Furthermore, DENV-2 co-localized with clathrin on the cell surface but did not enter the PCZ-treated cells (FIG. 3). Therefore, PCZ blocks clathrin-mediated endocytosis of DENV entry by affecting clathrin distribution and relocation.

PCZ Interferes with DENV-2 Binding onto the Cell Surface.

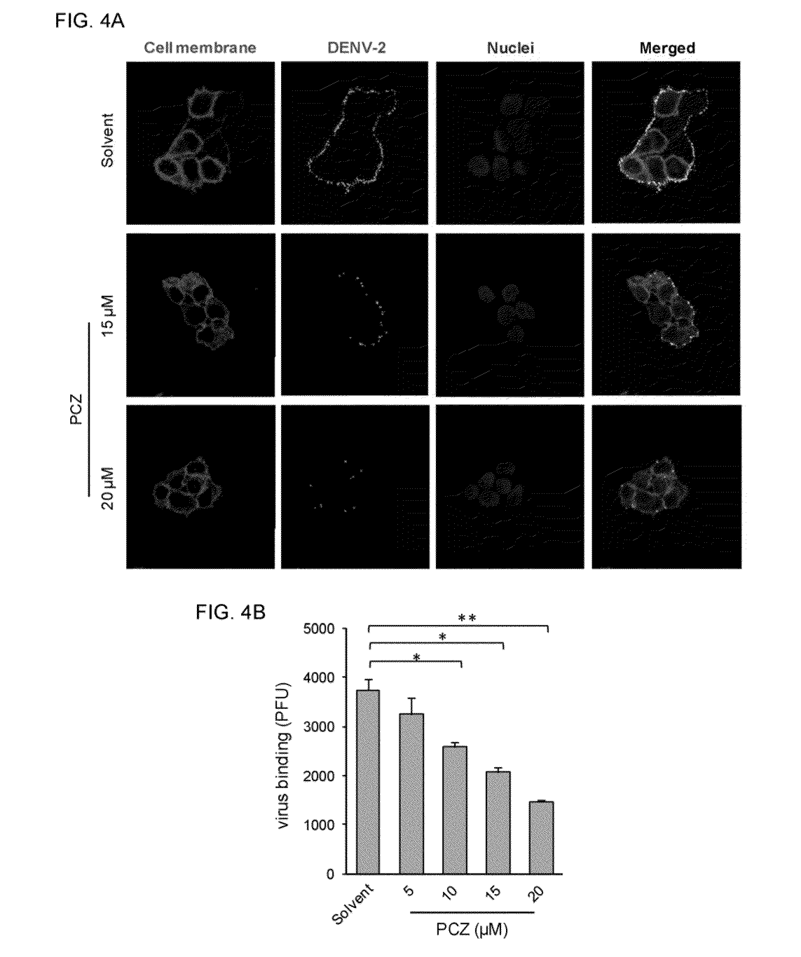

To address whether other steps of the viral life cycle are also targeted by PCZ, we performed a viral binding assay at 4° C. to evaluate the impact of PCZ on DENV binding. Confocal imaging revealed that fluorescent-labeled DENV-2 efficiently bound to the surface of solvent-treated HEK293T cells (FIG. 4A). However, PCZ treatment 1 h before and during virus adsorption at 4° C. reduced the levels of DENV-2 bound to the cell surface. This result was confirmed by quantification of viral binding by plaque forming assay; PCZ dose-dependently reduced DENV-2 binding to A549 cells (FIG. 4B). Therefore, PCZ interferes with DENV-2 binding and attachment to the cell surface.

The Inhibitory Effect of PCZ on DENV-2 Binding is Associated with Dopamine D2 Receptor (D2R).

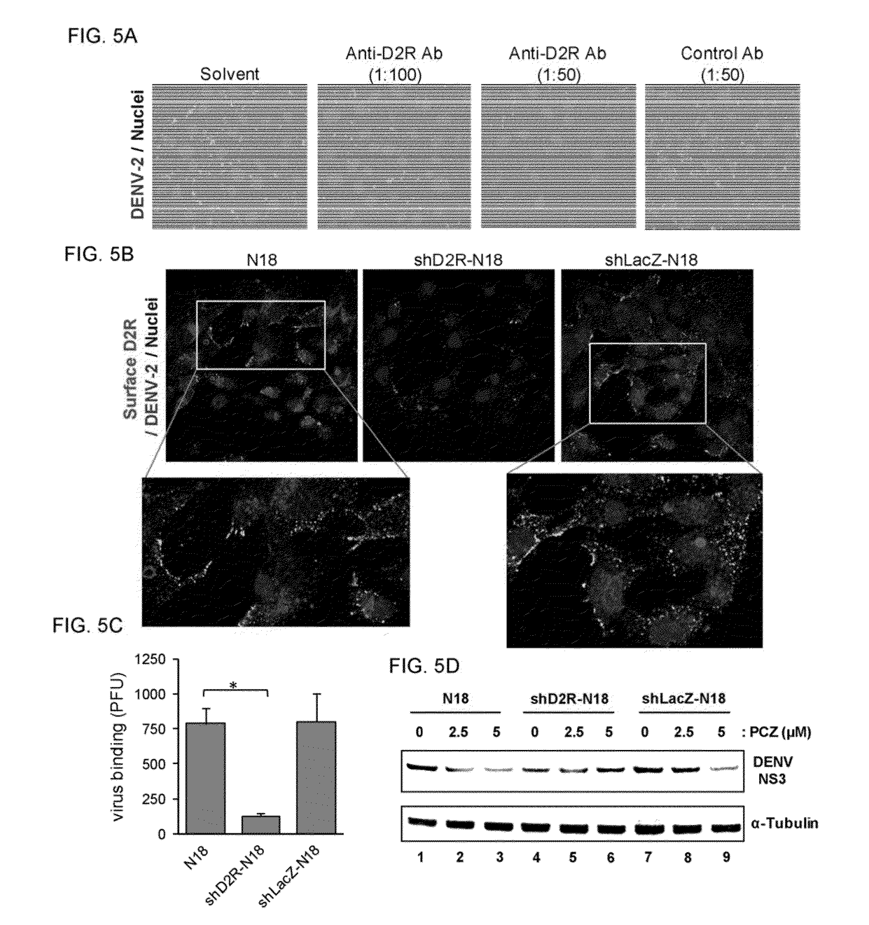

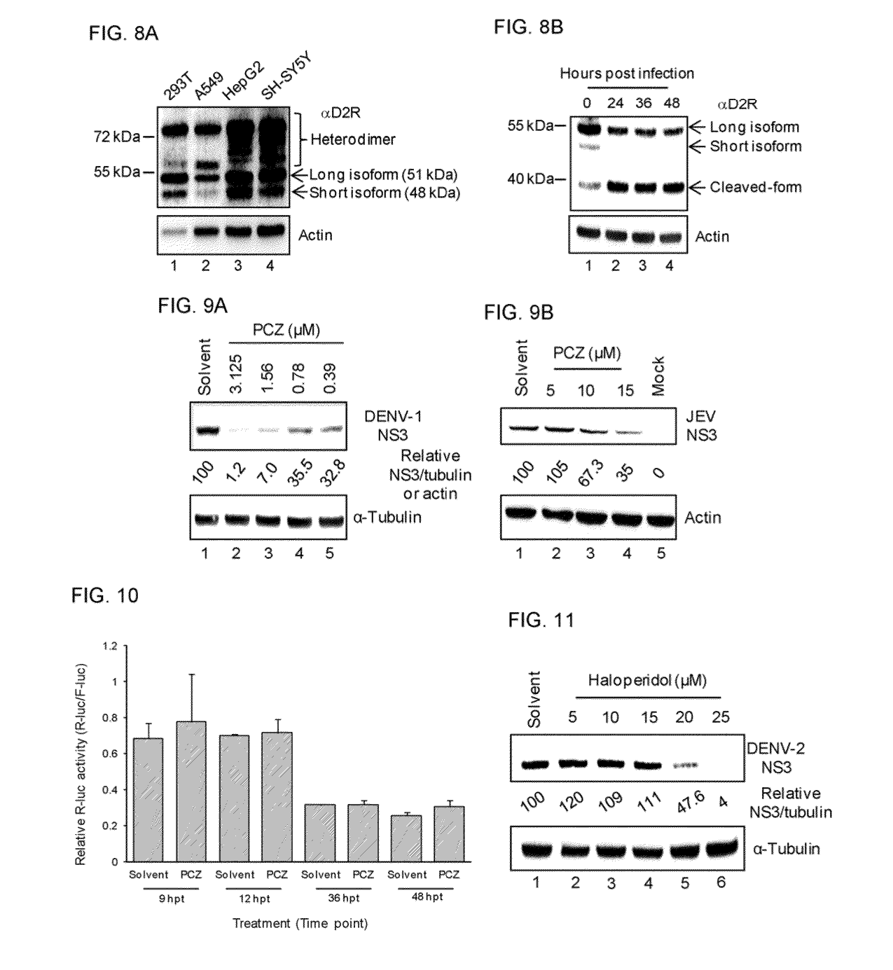

PCZ is a dopamine receptor antagonist and shows increased affinity for D2R. D2R is widely expressed in various cells and tissues, including neuronal cells, immune cells, and blood vessels. The cell lines we used also expressed. D2R (FIG. 8). To test whether the inhibitory effect of PCZ on DENV binding may be associated with D2R, we first determined whether D2R contributes to DENV binding by an antibody blockage experiment. Treatment with anti-D2R antibody but not the solvent or control antibody dose-dependently repressed DENV-2 binding (FIG. 5A). Next, we established stable D2R-deficient N18 cells by transduction with a lentivirus expressing shRNA targeting D2R, then examined whether D2R contributes to DENV-2 binding. Fluorescent-labeled DENV-2 largely colocalized with D2R on the surface of N18 cells and knockdown control shLacZ-N18 cells (FIG. 5B); shD2R-N18 cells lacking D2R expression showed less DENV-2 binding than controls (FIG. 5B). Quantification of the cell-bound virus by plaque-forming assay showed approximately 6 times lower DENV-2 binding with shD2R-N18 than controls (FIG. 5C). Finally, we tested whether the inhibitory effect of PCZ on DENV binding depended on D2R. Cells were pretreated with PCZ for 30 min, then DENV-2 was adsorbed at 4° C. for 2 h with various doses of PCZ. After extensive washing, cells were incubated overnight at 37° C. without PCZ. The antiviral effect of PCZ was noted in N18 and shLacZ-N18 but not shD2R-N18 cells (FIG. 5D). Thus, the inhibitory effect of PCZ on DENV binding is associated with D2R.

PCZ Protects Against DENV-2-Induced Lethality in Animal Models.

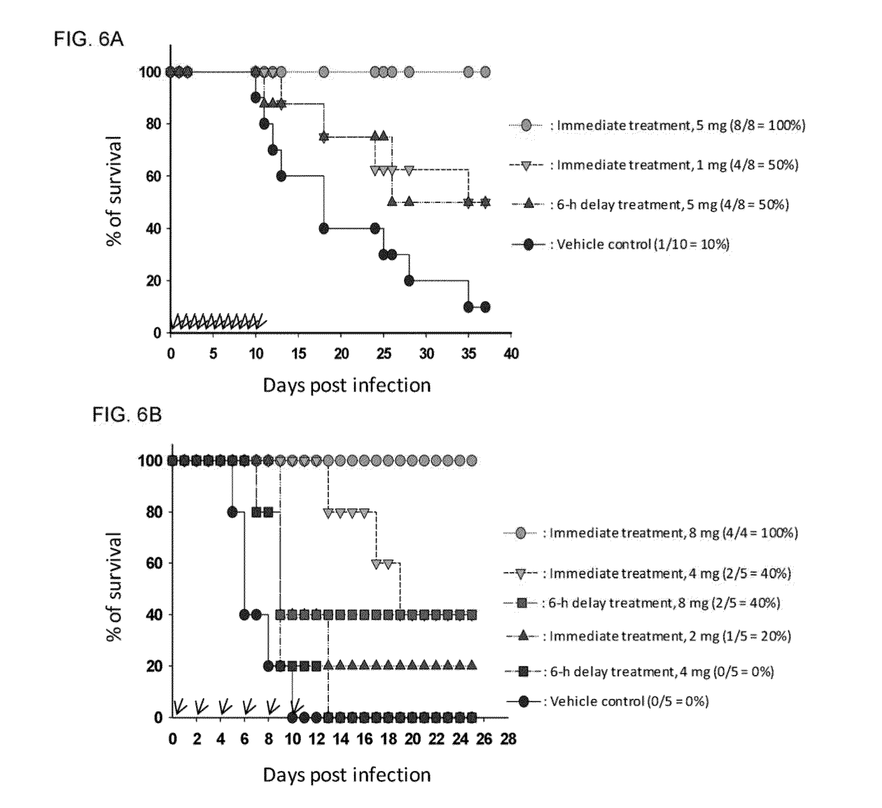

We then used a mouse challenge model to test whether PCZ exhibits in vivo antiviral property. STAT1 is a transcription factor activated by many cytokines, and disruption of the mouse Stat1 gene resulted in compromised innate immunity against viral diseases. We previously developed a DENV model in Stat1−/− mice that showed paralysis, hemorrhage, vascular leakage and death after challenge with a mouse-adapted DENV-2 strain. PCZ is available for the ingestion route (tablet or capsule) and parenteral route (injection), so we tested both routes, with PCZ-dimaleate and PCZ-Novamin, respectively, in our animal model. Stat1^{-/-} mice challenged with 5×10⁴ PFU/mouse of DENV-2 NGC-N strain received PCZ-dimaleate solution (1 and 5 mg PCZ/kg body weight/day) orally for 10 days, except for the first treatment was given by intraperitoneal (i.p.) injection, starting either immediately or at 6-h after DENV-2 infection for the prophylactic and therapeutic modes of treatment, respectively. A high dose of immediate PCZ treatment (5 mg/kg body weight/day) completely protected against death with DENV-2 infection, but 90% of the vehicle control mice died (FIG. 6A). Immediate treatment with a lower dose (1 mg PCZ/kg body weight/day) delayed animal mortality, with a moderate effect on overall animal survival (50%). In the therapeutic model (6-h delay group), PCZ treatment (5 mg PCZ/kg body weight) starting from 6 h post-infection for 10 days delayed death and improved the overall survival (50%) of DENV-2-inoculated animals.

We further evaluated the anti-DENV efficacy of PCZ-Novamin administered by i.p. route with a 2-day interval. After 10 days of infection, all mice in the vehicle control group died with. DENV-2 challenge, but immediate treatment with 8 or 4 mg PCZ/kg body weight/2 days completely protected mice against DENV-2 challenge (FIG. 6B). Even after the treatment stopped at day 10 post-infection, mice receiving a high dose (8 mg) of PCZ remained healthy, whereas some of the mice receiving a lower dose (4 mg) died, with overall survival 40%. An even lower dose, 2 mg PCZ/kg body weight/2 days, slightly delayed the DENV-2-induced lethality, with overall survival 20%. In the therapeutic mode of treatment, from 6 h post-infection and ending at day 10 post-infection, 40% of mice receiving 8 mg PCZ/kg body weight/2 days survived the DENV-2 challenge. A lower dose (4 mg kg body weight/2 days) of PCZ did not improve the overall survival but delayed the mortality. Thus, PCZ has a dose-dependent protective effect on DENV-2 challenge in delaying the lethality and improving overall survival.

### Discussion

Taking into accounts that: (1) PCZ showed anti-HCV activity in cell-based screening; (2) HCV and DENV, members of Flaviviridae, share similarities in life cycles and host-pathogen interaction profiles; (3) PCZ may relieve symptoms associated with DENV infection such as headache, nausea and vomiting; and (4) PCZ is a clinically approved drug available for human use, we selected PCZ to test its potential as an accelerated anti-DENV drug candidate. Indeed, PCZ exhibited in vitro and in vivo antiviral activity against DENV-2 infection. Interestingly, PCZ also suppressed the infection of other flaviviruses, such as DENV-1 and Japanese encephalitis virus (JEV), in cell culture assays (FIG. 9). Thus, PCZ may have a broad antiviral potential against members of Flaviviridae family.

Regarding the mode of action, PCZ likely targets 2 steps of DENV life cycle, binding and entry, but does not affect viral translation and RNA replication evaluated by using a DENV-2 replicon system (FIG. 10). Chlorpromazine, a phenothiazine with similar chemical structure as PCZ, inhibits clathrin-dependent endocytosis. We also found that PCZ blocks DENV entry by disrupting clathrin distribution (FIGS. 2 and 3). Interestingly, we found that DENV binding was affected by PCZ in a D2R-dependent manner, because DENV binding to the cell surface could be blocked by anti-D2R antibody and knockdown of D2R expression (FIGS. 4 and 5). To support the notion that dopamine receptor blockage reduces DENV infection, we further tested another D2R antagonist with unknown effect on clathrin, haloperidol, for its antiviral potential; haloperidol also showed antiviral activity against DENV-2 infection (FIG. 11). Thus, our data strongly argue for the involvement of D2R in DENV infection. However, the detailed mechanism such as whether D2R functions as a DENV receptor or facilitates DENV binding to its putative receptor remains elusive. Since PCZ targets two distinct cellular components, instead of on the virus, it is less likely to develop drug-resistant virus strains with PCZ treatment.

Dopamine receptors belong to the 7-transmembrane domain G-protein coupled receptor family and are classed as D1-like receptors (D1R and D5R subtypes) or D2-like receptors (D2R, D3R and D4R subtypes). Since PCZ and haloperidol are antagonists mainly targeting D2R among the dopamine receptors, D2R is more likely to participate in DENV infection. However, the potential role of other subtypes of dopamine receptors in DENV infection remains to be studied. We noted a reduced full-length D2R protein band and appearance of a smaller D2R-related protein band in DENV-2 infected cells (FIG. 8B), which suggests degradation of D2R during DENV infection. Further understanding the signaling event and implication of the D2R pathway in DENV infection might shed light on dengue pathogenesis and reveal new drug targets.

The US Food and Drug Administration lists PCZ as an active pharmaceutical ingredient approved as an abbreviated new drug application, which conveys its safety information and pharmacodynamics in both humans and animals. In the material safety data sheets for PCZ (Sigma and Science Lab.com), the LD₅₀ in mice has been described as 191 and 400 mg/kg body weight for i.p. injection and oral route, respectively. Thus, the doses of PCZ showing in vivo protective effect against DENV-2 infection in our animal model (FIG. 6) are well below the potentially hazardous doses. The human equivalent dose of 5 mg PCZ/kg day for mice is 0.405 mg/kg/day for humans calculated by a published method; thus, for a 60-kg person, the dose would be 24.3 mg/day. The clinically recommended dose of PCZ-maleate to prevent nausea and vomiting is 5 to 10 mg/dose 2 to 3 times daily and for treating nausea and vomiting is 20 mg then 10 mg 2 h later if required. Therefore, to reach the noted protective dose of PCZ in humans is feasible.

So far, a specific antiviral agent blocking DENV infection is lacking, and supportive care and symptomatic treatments are commonly used to treat dengue patients. Our finding that PCZ, a drug widely used to relieve anxiety, headache, nausea and vomiting, common symptoms among dengue patients, has potent antiviral activity against DENV infection is novel and significant. PCZ treatment in dengue patients might have 2 beneficial effects: reducing DENV infection and relieving clinical symptoms. Because dengue is an acute disease, the window and length of therapeutic treatment is narrow and short. A clinically used compound showing anti-DENV activity such as PCZ might then be prophylactically prescribed to high-risk individuals and/or patients with fever before dengue diagnosis in epidemic areas during dengue outbreaks. Another potential advantage of PCZ treatment in DF patients is to prevent the development of DHF and DSS, because a higher magnitude of DENV replication is associated with severe dengue disease. Overall, we reveal that PCZ has a previously unknown antiviral function and could be further clinically evaluated as a potential therapeutic as well as intervention in DENV infection.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. A method of preventing and/or treating flavivirus infection, comprising:
administering to a subject in need thereof a composition comprising:
a) a dopamine D2 receptor antagonist in an amount effective for preventing and/or treating flavivirus infection; and
b) a pharmaceutically acceptable carrier;
wherein the dopamine D2 receptor antagonist exhibits an antiviral activity in inhibiting dengue virus replication and/or inhibiting nonstructural protein 3 (NS 3) production in a host cell.

2. The method of claim 1, wherein the dopamine D2 receptor antagonist is at least one selected from the group consisting of prochlorperazine or a salt thereof, and haloperidol.

3. The method of claim 1, wherein the flavivirus is selected from the group consisting of a dengue virus, an encephalitis virus, and a West Nile virus.

4. The method of claim 3, wherein the encephalitis virus is a Japanese encephalitis virus.

5. The method of claim 3, wherein the dengue virus is serotype 2 or serotype 1.

6. The method of claim 1, wherein the salt of prochlorperazine is selected from the group consisting of prochlorperazine maleate and prochlorperazine dimethanesulfonate.

7. The method of claim 1, wherein the subject is a high-risk human or a patient with fever before dengue diagnosis in epidemic areas during dengue outbreaks.

8. The method of claim 1, wherein the subject is a patient with dengue fever.

9. The method of claim 1, wherein the amount of prochlorperazine, or a salt thereof, is effective in inhibiting flavivirus binding to and/or entry into the cells of the subject.

10. The method of claim 1, wherein the amount of prochlorperazine, or a salt thereof, is effective in inhibiting flavivirus protein synthesis or flavivirus replication in the cells of the subject.

11. The method of claim 1, wherein the composition is a tablet, capsule or injection dosage form.

12. A method of preventing development of dengue hemorrhagic fever and/or dengue shock syndrome, comprising:
administering to a dengue fever patient a composition comprising:
a) a therapeutically effective amount of dopamine D2 receptor antagonist; and
b) a pharmaceutically acceptable carrier;
wherein the dopamine D2 receptor antagonist exhibits an antiviral activity in inhibiting dengue virus replication and/or inhibiting nonstructural protein 3 (NS3) production in a host cell.

13. The method of claim 12, wherein the dopamine D2 receptor antagonist is at least one selected from the group consisting of prochlorperazine or a salt thereof, and haloperidol.

14. The method of claim 12, wherein the salt of prochlorperazine is selected from the group consisting of prochlorperazine maleate and prochlorperazine dimethanesulfonate.

15. The method of claim 12, wherein the dengue virus is serotype 2 or serotype 1.

16. A method of preventing and/or treating dengue virus infection, comprising:
administering to a subject in need thereof a composition comprising:
a) a dopamine D2 receptor antagonist in an amount effective for preventing and/or treating dengue virus infection; and
b) a pharmaceutically acceptable carrier.

17. The method of claim 16, wherein the dopamine D2 receptor antagonist is at least one selected from the group consisting of prochlorperazine or a salt thereof, and haloperidol.

18. The method of claim 17, wherein the salt of prochlorperazine is selected from the group consisting of prochlorperazine maleate and prochlorperazine dimethanesulfonate.

19. The method of claim 1, wherein the administering step administers to a human prochlorperazine at least 0.4 mg/kg/day.

20. The method of claim 1, wherein the administering step is performed by injection every 2 days for at least 10 days.


SEQUENCE LISTING (2 sequences)

SEQ ID NO: 1 - DNA, 21 bp, Artificial Sequence
  shRNA targeting D2 receptor
ccactacaactactatgccat

SEQ ID NO: 2 - DNA, 21 bp, Artificial Sequence
  shRNA targeting LacZ
ccactacaactactatgccat